(19) United States
(12) Patent Application Publication
(10) Pub. No.: US 2014/0127185 A1
(43) Pub. Date: May 8, 2014

(54) METHODS AND COMPOSITIONS USING NEUROPROTECTIVE STEROIDS AND THROMBOLYTIC AGENTS

(71) Applicant: Emory University, Atlanta, GA (US)

(72) Inventors: Donald G. Stein, Atlanta, GA (US); Iqbal Sayeed, Atlanta, GA (US)

(73) Assignee: Emory University, Atlanta, GA (US)

(21) Appl. No.: 14/068,475

(22) Filed: Oct. 31, 2013

Related U.S. Application Data
(60) Provisional application No. 61/721,779, filed on Nov. 2, 2012.

(51) Int. Cl. A61K 38/48; A61K 31/57

(52) U.S. Cl. CPC A61K 38/482; A61K 31/57
USPC 424/94.64

(57) ABSTRACT

Described herein are compositions and methods for treating or preventing ischemic injury. In particular, the methods and compositions relate to the use of a neuroprotective steroid, such as progesterone or allopregnanolone, and a thrombolytic agent, such as tissue plasminogen activator (tPA). Also described are compositions and methods for reducing the risks of thrombolytic agent treatment using a neuroprotective steroid.

This application claims priority to U.S. Provisional Application 61/721,779, filed on Nov. 2, 2012, the contents of which are incorporated herein in their entirety.

### FIELD

Described herein are methods and compositions using a neuroprotective steroid and a thrombolytic agent for treating ischemic injury, such as may be associated with ischemic stroke. Also described are compositions and methods for reducing the risks of thrombolytic agent treatment using a neuroprotective steroid.

### BACKGROUND

Ischemic injury occurs when there is a restriction in blood flow to tissues, and may be associated with various conditions, such as a neurodegenerative reaction to injury or disease, traumatic brain injury, ischemic CNS injury, spinal cord injury, ischemic stroke, and anterior optic nerve ischemic injury. For example, ischemic stroke occurs when a blood vessel that brings oxygen and nutrients to the brain is clogged by a blood clot or some other mass.

Some studies indicate that acute inflammatory response contributes significantly to injury after ischemia. See, e.g., Perera, et al, J. Clin. Neurosc., 2005, 13:1-8. The stroke process triggers an inflammatory reaction that may last up to several months. Infiltrating leukocytes are thought to contribute to secondary ischemic damage by producing toxic substances that kill brain cells and disrupt the blood-brain barrier (BBB). See del Zoppo, et al, Thromb Res., 2000, 98:73-81. Infiltration occurs when leukocytes bind endothelial intercellular adhesion molecule-1 (ICAM-I) and ICAM-I is upregulated after ischemia.

Thrombolytic agents such as tissue plasminogen activator (tPA), streptokinase, urokinase and desmoteplase may be used to treat ischemic injury. However, thrombolytic agents carry their own risks, such as risks of bleeding and hemorrhagic injury. Despite its risks, tPA is the only treatment for acute stroke approved by the United States Food and Drug Administration. A serum protease, tPA, acts specifically on serum plasminogen, an inactive protease circulating in the blood, which is converted to active plasmin by tPA. The proteolytic activity of the tPA/plasminogen system allows for clearance of the flow-obstructing clots and restoration of blood flow to the oxygen-deprived areas of the brain. A typical protocol for ischemic stroke requires an intravenous administration of up to 90 mg of tPA per patient within the first three hours of stroke. The use of tPA comes with neurovascular complications, including increasing the risk of intracerebral or intracranial hemorrhage. Such hemorrhage hampers the widespread use of tPA as a therapeutic. See Lapchak, Expert Opin. Investig. Drugs, 2002, 11: 1623-1632. Fewer than 5% of stroke victims can be treated with tPA. See Grotta et al, Arch Neurol., 2001; 58(12): 2009-13; Goldstein, Circulation, 2007, 116(3): 1504-14. tPA is also cytotoxic, increasing the permeability of the neurovascular unit and leading to the development of brain edema. Although the mechanisms underlying tPA's neurovascular complications are not fully understood, some research has suggested that blood-brain barrier disruption (BBB) contributes to the complications. Hamann et al, J Cereb Blood Flow Metab, 1996; 16(6): 1373-8. Similar mechanisms of action and risks are associated with other thrombolytic agents.

### SUMMARY

Described herein are methods of treating a patient suffering from or at the risk of ischemic injury, comprising administering to the patient: (i) a neuroprotective steroid or a pharmaceutically acceptable salt, ester or prodrug thereof, and (ii) a thrombolytic agent. In specific embodiments, the steroid is a progestogen. In specific embodiments, the steroid is selected from the group consisting of progesterone and allopregnanolone. In specific embodiments, the thrombolytic agent is selected from the group consisting of tPA, streptokinase, urokinase and desmoteplase.

In some embodiments, the steroid and thrombolytic agent are administered in separate compositions. In other embodiments, the steroid and thrombolytic agent are administered in the same composition. In specific embodiments, the steroid and thrombolytic agent are administered simultaneously. In other specific embodiments, the steroid and thrombolytic agent are administered sequentially. In other specific embodiments, the steroid is administered before the thrombolytic agent is administered.

In some embodiments, the ischemic injury is associated with a condition selected from the group consisting of a neurodegenerative reaction to injury or disease, traumatic brain injury, ischemic CNS injury, spinal cord injury, ischemic stroke, and anterior optic nerve ischemic injury. In specific embodiments, the ischemic injury is associated with ischemic stroke.

Also described are methods of reducing the risks of treatment with a thrombolytic agent, comprising administering a neuroprotective steroid or a pharmaceutically acceptable salt, ester or prodrug thereof to a patient who has been or will be administered a thrombolytic agent,

Also described are pharmaceutical compositions comprising: (a) a neuroprotective steroid or a pharmaceutically acceptable salt, ester or prodrug thereof; and (b) a thrombolytic agent, optionally, in a pharmaceutically acceptable carrier. In some embodiments, the steroid is a progestogen. In some embodiments, the steroid is selected from the group consisting of progesterone and allopregnanolone. In some embodiments, the thrombolytic agent is selected from the group consisting of tPA, streptokinase, urokinase and desmoteplase. In some embodiments, the composition is formulated for intravenous administration.

In some embodiments, the composition is for use in treating a patient suffering from or at the risk of ischemic injury. In specific embodiments, the ischemic injury is associated with a condition selected from the group consisting of a neurodegenerative reaction to injury or disease, traumatic brain injury, ischemic CNS injury, spinal cord injury, ischemic stroke, and anterior optic nerve ischemic injury. In specific embodiments, the ischemic injury is associated with ischemic stroke.

Also described is the use of a composition as described herein, in the preparation of a medicament for treating a patient suffering from or at the risk of ischemic injury.

Also described is a pharmaceutical composition comprising a neuroprotective steroid or a pharmaceutically acceptable salt, ester or prodrug thereof, optionally, in a pharmaceutically acceptable carrier, for use in reducing the risks of treatment with a thrombolytic agent.

Also described is the use of a composition comprising a neuroprotective steroid or a pharmaceutically acceptable salt, ester or prodrug thereof, optionally, in a pharmaceutically acceptable carrier, in the preparation of a medicament for reducing the risks of treatment with a thrombolytic agent.

### BRIEF DESCRIPTION OF THE DRAWINGS

FIG. 1 shows data indicating that progesterone and allopregnanolone attenuate MMP-9 and MMP-2 induction by tPA. (“S”=sham, “L”=lesion, “LP”=lesion+progesterone and “LA”=lesion+allopregnanolone.) FIG. 1A shows Western blot analysis of MMP-9 and MMP-2 FIG. 1B shows densitometric analysis of MMP-9 and MMP-2. FIGS. 1C and 1D show gelatin zymography data of MMP-9 and MMP-2. FIG. 1E shows immunohistochemical samples of MMP-9 and MMP-2 expression.

FIG. 2A shows the effects of tPA and progesterone on hemorrhage volume at 24 hours post-stroke (reported as mg hemoglobin, mean±SE), and shows that delayed tPA (e.g., tPA administered at 4.5 hours) induced cerebral hemorrhage, while combination therapy with delayed tPA plus progesterone prevented hemorrhage. FIG. 2B shows the effects of tPA and progesterone on infarct volume at 24 hours post-stroke, and shows that the combination therapy reduced infarct volume compared to tPA-only treatment. FIG. 2C shows the effect of combination therapy with tPA and progesterone on brain swelling at 24 hours post-stroke, and shows that the combination therapy reduced the brain swelling that was associated with tPA treatment.

FIG. 3 shows the effect of progesterone on transendothelial electrical resistance (TEER) following in vitro BBB dysfunction induced by hypoxia and reperfusion (H/R) and tPA treatment. FIG. 3A shows that combination therapy with tPA plus progesterone prevented tPA-associated increase in IgG (reported as mean±SEM). FIG. 3B shows that the TEER (reported as Ω·cm², mean±SEM; #P<0.01 v. H/R (t-test, n=5-6)) was decreased by H/R, and that the decrease was significantly prevented by pretreatment with 5, 10 or 20 μM of progesterone. FIG. 3C shows that pre-treatment with progesterone prevented H/R-induced and tPA-associated reduction in TEER (reported as Ω·cm², mean±SEM; #P<0.05 v. H/R+PROG; #P<0.05 v. tPA alone; #P<0.05 v. tPA+PROG (n=5, 6)).

FIG. 4 shows the effect of progesterone against tight junction proteins (ZO-1, occludin and claudin-5) at 24 hours after ischemia, using densitometri analysis of immunoreactive bands normalized to β-actin as loading control. Results are reported as mean±SEM; #P<0.05 v. control; *P<0.05 v. tPA plus progesterone. Western blot images and quantitative data showing levels of ZO-1, occludin and claudin-5 in vivo (FIGS. 4A-C) or in vitro (FIGS. 4D-F) are shown.

FIG. 5 shows the effect of progesterone on MMP-9 activity 24 hours after ischemia, as revealed by fluorometric assay of lysates from control or ischemic cortices using fluorescence resonance energy transfer (FRET) peptides as substrates (n=6-7). FIG. 5A shows that progesterone inhibited MMP-9 expression induced by tPA treatment following stroke. FIG. 5B shows that the increased in MMP-9 levels in brain endothelial cells exposed to H/R and tPA was completely inhibited when the cells were pretreated with progesterone.

### DETAILED DESCRIPTION

Described herein are methods and compositions using a neuroprotective steroid and a thrombolytic agent for treating ischemic injury, such as may be associated with ischemic stroke. Also described are compositions and methods for reducing the risks of thrombolytic agent treatment, such as tPA treatment, using a neuroprotective steroid, such as progesterone or allopregnanolone.

### I. DEFINITIONS

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the disclosure. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “patient” as used herein includes any animal, including humans. In particular, the term is intended to identify those animals in need of the treatments and compositions described herein, whether to treat disease or injury, prevent disease or injury, or maintain health. Although in many embodiments the patient is a human, other animals and in particular mammals are also encompassed by the disclosure.

### II. NEUROPROTECTIVE STEROIDS

Neuroprotective steroids useful herein include progesterone and prodrugs, metabolites and analogues of progesterone; analogues of progesterone metabolites or derivatives, and other non-progestin steroid compounds or analogues or secosteroid analogues that exhibit in vivo efficacy in the methods described herein. Exemplary neuroprotective steroids include those described herein and in U.S. publication 2011/0263553 and PCT publication WO 2009/108804, each of which is incorporated herein by reference in its entirety. For example, progestins useful in the methods and compositions described herein include those set forth in WO 2010/088409 (the entire contents of which are incorporated by reference in its entirety) and stereoisomers thereof, including those listed at pages 22-60.

In some specific embodiments, the neuroprotective steroid is progesterone.

In some specific embodiments, the neuroprotective steroid is allopregnanolone, a principal metabolite of progesterone. Allopregnanolone is a potent, positive modulator of CNS GABA, receptor functions. Orchinik et al, Brain Res, 1994, 646(2): 258-66; Paul et al, Faseb J, 1992, 6(6): 2311-22.

In particular embodiments, the neuroprotective steroid is represented by formula (I):

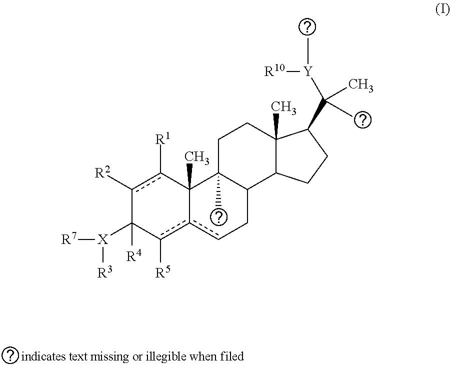

wherein X is O, N or S;

Y is O, N or S;

R¹, R², R⁵ and R⁶ are independently hydrogen, alkyl, halogen, hydroxyl cycloalkyl, cycloalkenyl, alkenyl, alkynyl, aryl, alkylaryl, arylalkyl, heterocyclic, heteroaryl, amino, thiol, alkoxy, sulfide, nitro, cyano, azide, sulfonyl, acyl, carboxyl, an ester, an amide, carbamate, carbonate, an amino acid residue or a carbohydrate;

R⁴ is hydrogen or alkyl; or R⁴ and R⁷ together form a double bond;

R³ is hydrogen, optionally substituted acyl, a residue of an amino acid, a carbohydrate, —OR¹¹, —NR¹¹R¹² or R³ is absent;

R⁷ is hydrogen or is absent, or R⁷ together with R⁴ forms a double bond;

R⁸ is hydrogen, optionally substituted acyl, a residue of an amino acid, a carbohydrate, —OR¹¹, —NR¹¹R¹² or R⁸ absent;

R⁹ is hydrogen or alkyl; or R⁹ and R¹⁰ together form a double bond;

R¹⁰ is hydrogen or is absent, or R¹⁰ together with R⁹ forms a double bond;

R¹¹ is the residue of an amino acid, a carbohydrate or an optionally substituted ester or a substituted acyl;

R¹² is hydrogen or alkyl; and

the dotted line indicates the presence of either a single bond or a double bond, wherein the valences of a single bond are completed by hydrogens,

provided that

at least one of XR³R⁷ or YR⁸R¹⁰ is not ═O or OH, and that if the dotted line between C4 and C5 or between C5 and C6 represents a double bond then the other dotted line between C4 and C5 or between C5 and C6 represents a single bond; and with the proviso that neither XR³R⁷ nor YR⁸R¹⁰ represents an ester of aspartic acid, glutamic acid, gama amino butyric acid or a-2-(hydroxyethylamino)-propionic acid; and

with the proviso that when Y is N, R⁸ does not represent aspartic acid, glutamic acid, gama amino butyric acid or a-2-(hydroxyethylamino)-propionic acid.

In some embodiments, the neuroprotective steroid is a progestogen. Progestogens include, for example, derivatives of progesterone such as 5-α-dihydroprogesterone, 6-dehydro-retroprogesterone (dydrogesterone), hydroxyprogesterone caproate, levonorgestrel, norethindrone, norethindrone acetate; norethynodrel, norgestrel, medroxyprogesterone, chlormadinone, and megestrol. “Progestogen” also includes, but is not limited to, modifications that produce 17α-OH esters of progesterone, as well as, modifications that introduce 6-α-methyl, 6-methyl, 6-ene, and 6-chloro substituents onto progesterone, and/or 19-norprogesterones. Further, non-limiting examples, of synthetic progestogens include, norethindrone (Micronor®), norgestrel (Ovrette®), levonorgestrel (Norplant®; with ethinyl estradiol; Alesse®, Nordette®), gestodene, medroxyprogesterone acetate (Provera®), promegestone, nomegestrol acetate, lynestrenol and dienogest.

In some embodiments, the neuroprotective steroid is selected from the group consisting of progesterone, norethynodrel, norethidrone acetate, medroxyprogesterone, medroxyprogesteron 17-acetate, levonorgestrel, dydrogesterone, hydroxyprogesterone caproate, norethidrone, gestodene, nomegestrol acetate, promegestone, dienogest, chlormadinion, megestrol, megestrol acetate, and/or mixtures thereof.

In some embodiments the neuroprotective steroid is selected from the group consisting of 5-α-dihydroprogesterone, medroxyprogesterone, dydrogesterone, and progesterone and/or mixtures thereof.

As noted above, in specific embodiments, the neuroprotective steroid is progesterone. The term “progesterone” as used herein refers to a member of the progestogen family having the structure of Formula II below:

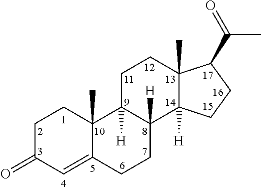

Progesterone is also known as D4-pregnene-3,20-dione; delta-4-pregnene-3,20-dione; or pregn-4-ene-3,20-dione.

As noted above, in specific embodiments, the neuroprotective steroid is allopregnanolone. Allopregnanolone also is known as 3α-hydroxy-5α-pregnan-20-one or 3α,5α-tetrahydroprogesterone, and has the structure of Formula III below:

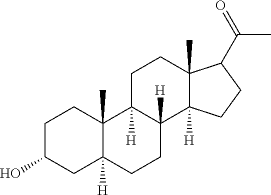

The neuroprotective steroid can be administered as a “prodrug.” As used herein, a “prodrug” is an inactive or less active form of the neuroprotective steroid that, once administered, is metabolized in vivo into an active or more active form of the neuroprotective steroid. In some embodiments, the prodrug is oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active drug form. In other embodiments, the prodrug is a compound that has at least one biologically labile protecting group on a functional moiety of the compound, which protecting group is removed in vivo. A number of prodrug ligands are described in Jones and Bischofberger, Antiviral Research, 1995, 27: 1-17. In specific embodiments, alkylation, acylation or other lipophilic modification of the steroid will increase the stability of the steroid. Examples of substituent groups that can replace one or more hydrogens on the steroid are alkyl, aryl, steroids, carbohydrates, including sugars, 1,2-diacyl glycerol and alcohols.

The neuroprotective steroid can be administered as a pharmaceutically acceptable salt or ester.

### III. THROMBOLYTIC AGENTS

As used herein, the term “thrombolytic agent” includes but is not limited to thrombolytic agents such as tissue plasminogen activator (tPA), streptokinase, urokinase and desmoteplase, which are used (or being developed) to treat ischemic injury.

tPA is a serum protease acting specifically on serum plasminogen. tPA also is known as alteplase and is available commercially as Activase® (Genentech). The tPA in Activase® is a recombinant glycoprotein having 527 amino acid residues. Activase® is provided as a lyophilized powder comprising tPA, L-arginine, phosphoric acid, and polysorbate 80, and is reconstituted with sterile water for injection prior to use. tPA can be isolated from animal sources (including human sources) or produced recombinantly.

Streptokinase is a protein that acts to convert plasminogen to plasmin. Streptokinase is commercially available in some countries as Streptase® (Aventis Behring). Streptase® is provided as a lyophilized white powder comprising streptokinase, cross-linked gelatin polypeptides, sodium L-glutamate, sodium hydroxide and albumin, and is reconstituted and diluted for injection prior to use. The streptokinase in Streptase® is derived from streptococci bacteria. Streptokinase also can be produced recombinantly.

Urokinase is a protein that converts plasminogen to plasmin. Urokinase is commercially available in some countries as Abbokinase® (Abbott) and Kinlytic™ (Microbix). Both of these commercial compositions contain the low molecular weight form of urokinase, consisting of an A chain of 2,000 daltons linked by a sulfydryl bond to a B chain of 30,400 daltons. Abbokinase® and Kinlytic™ are provided as a lyophilized white powder containing 250,000 IU urokinase per vial, mannitol, albumin and sodium chloride. Prior to use, both of these commercial compositions are reconstituted with sterile water to produce from 50,000 to 750,000 International Units (IU) of urokinase activity per mL, 0.5% mannitol, 5% albumin and 1% sodium chloride. The urokinase in Abbokinase® and Kinlytic™ is obtained from human neonatal kidney cells grown in tissue culture. Urokinase also can be produced recombinantly.

Desmoteplase is a thrombolytic enzyme that converts plasminogen into plasmin. Desmoteplase is currently undergoing clinical trials as DIAS-3 and DIAS-4 (Lundbeck), which are recombinantly produced. Desmoteplase also can be isolated from the vampire bat Desmondus Rotundus.

### IV. PHARMACEUTICAL COMPOSITIONS

The neuroprotective steroid and thrombolytic agent can be administered in the same or separate pharmaceutical compositions. The pharmaceutical composition(s) can include a pharmaceutically acceptable carrier or diluent as is known in the art. For example, the pharmaceutically acceptable carrier may be inorganic or organic, solid or liquid, a particulant, a powder, a solution, a suspension or an emulsion. Suitable vehicles and their formulation are described, for example, in REMINGTON'S PHARMACEUTICAL SCIENCES (16th ed.) (A. Osol, ed. Mack Publishing Co., Easton Pa., 1980).

In some embodiments, one or more of the compositions is a solution or suspension for parenteral, intramuscular, intradermal, subcutaneous, or topical application. Such a composition can include excipients known for use in such dosage forms, such as a sterile diluent such as water, saline, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In some embodiments, one or more of the compositions is a parental preparation and is optionally provided in ampoules, disposable syringes or multiple dose vials made of glass or plastic.

In specific embodiments, a composition suitable for parental administration comprises a sterile aqueous preparation of a thrombolytic agent and a neuroprotective steroid, which can be isotonic with the blood of the recipient patient.

In some embodiments, one or more of the compositions is in the form of a nasal spray formulation, and may comprise one or more preservative agents and isotonic agents. Such formulations may be adjusted to a pH and isotonic state compatible with the nasal mucous membranes.

In some embodiments, one or more of the compositions is in a form suitable for administration as an aerosol by inhalation. Such compositions may comprise solid particles of the active agent(s) suitable for inhalation. The composition may be provided in a small chamber and nebulized. Nebulization may be accomplished by compressed air or by ultrasonic energy to form a plurality of liquid droplets or solid particles comprising the compounds or salts.

In some embodiments, one or more of the compositions is an oral composition that includes an inert diluent or an edible carrier. In specific embodiments, the composition is enclosed in gelatin capsules or compressed into tablets. For example, the therapeutic agent(s) can be incorporated with excipients and prepared in the form of tablets, troches or capsules. Pharmaceutically compatible binding agents, and/or adjuvant materials suitable for use in such dosage forms are known in the art and can be included as part of the composition.

In some embodiments, one or more of the compositions is a controlled release composition. Controlled release preparations may be prepared using polymers to complex or absorb the therapeutic agent(s) to be delivered. The controlled delivery may be exercised by selecting appropriate macromolecules (for example, polyesters, polyamino acids, hydrogels, poly(lactic acid), polyvinyl pyrrolidone, ethylene-vinylacetate, methylcellulose, carboxymethylcellulose, or protamine sulfate). The rate of drug release may also be controlled by altering the concentration of such macromolecules. Alternatively, it is possible to entrap the therapeutic agents in microcapsules prepared, for example, by coacervation techniques or by interfacial polymerization, for example, by the use of hydroxymethyl cellulose or gelatin-microcapsules or poly(methylmethacrylate) microcapsules, respectively, or in a colloid drug delivery system, for example, liposomes, albumin, microspheres, microemulsions, nanoparticles, nanocapsules, or in macroemulsions. Such techniques are disclosed in Remington's Pharmaceutical Sciences (1980).

In some embodiments, one or more of the compositions comprises a liposomal formulation. When the compounds or salts thereof are an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer that forms the structure of the liposome. In either instance, the liposomes that are produced may be reduced in size, as through the use of standard sonication and homogenization techniques. The liposomal formulations containing the progesterone analogue or salts thereof, may be lyophilized to produce a lyophilizate which may be reconstituted with a pharmaceutically acceptable carrier, such as water, to regenerate a liposomal suspension.

In some embodiments the neuroprotective steroid is provided in a composition that includes a therapeutically effective amount for neuroprotection. For example, the neuroprotective steroid may be present at a dose of about 0.1 ng to about 100 g per kg of body weight, about 10 ng to about 50 g per kg of body weight, from about 100 ng to about 1 g per kg of body weight, from about 1 ug to about 100 mg per kg of body weight, from about 1 ug to about 50 mg per kg of body weight, from about 1 mg to about 500 mg per kg of body weight, or from about 1 mg to about 50 mg per kg of body weight. In other embodiments, the amount of neuroprotective steroid administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

In some embodiments the neuroprotective steroid is provided in a composition that includes an amount effective to achieve a serum level of neuroprotective steroid of about 100 ng/ml to about 2000 ng/ml, including about 100 ng/ml to about 1000 ng/ml, including about 200 ng/ml to about 450 ng/ml, such as about 350 ng/ml to about 450 ng/ml, as described, for example, in U.S. Pat. No. 7,473,687, the entire contents of which are incorporated herein by reference in its entirety.

In some embodiments the thrombolytic agent is provided in a composition suitable for intravenous administration, or in a composition to be diluted prior to intravenous administration. For example, as noted above, tPA, streptokinase and urokinase are available commercially for intravenous administration, while desmoteplase is currently being administered intravenously in clinical trials.

In some embodiments the thrombolytic agent is provided in a composition that includes a therapeutically effective amount of thrombolytic agent, such as a therapeutically effective amount in accordance with protocols approved by the U.S. Food and Drug Administration for the treatment of acute ischemic stroke or in clinical development for the treatment of ischemic stroke or other ischemic injury. For example, tPA may be provided in a composition formulated for intravenous administration to provide a dose of 0.9 mg/kg body weight of the patient, such as up to a total dose of 90 mg; streptokinase may be provided in a composition formulated for intravenous administration, such as to provide a dose of from 1,500,000 to 2,400,000 IU; urokinase may be provided in a composition formulated for intravenous administration, such as to provide a dose of from 50,000 IU to 750,000 IU, including 500,000 or 300,000 IU; desmoteplase may be provided in a composition formulated for intravenous administration, such as to provide 90 μg/kg, such as up to 9,000 μg (or 9 mg).

In some embodiments the thrombolytic agent and neuroprotective steroid are provided in a single composition, wherein the thrombolytic agent and the neuroprotective steroid may each be present in any of the amounts discussed above.

### V. METHODS OF TREATMENT

Described herein are methods for treating or reducing the risks and/or effects of ischemic injury. The methods involve administering a therapeutically effective amount of a neuroprotective steroid (or a pharmaceutically acceptable salt, ester or prodrug thereof), and a therapeutically effective amount of a thrombolytic agent.

Also described herein are methods for treating or reducing the risks and/or effects of thrombolytic agent treatment, such as cerebral hemorrhage and cerebral edema, such as may be observed with thrombolytic agent treatment after ischemic stroke. The methods involve administering a therapeutically effective amount of a neuroprotective steroid (or a pharmaceutically acceptable salt, ester or prodrug thereof) to a patient who has been, is, or will be administered a thrombolytic agent.

In accordance with any of these methods, the neuroprotective steroid and thrombolytic agent can be administered sequentially. In some embodiments, the neuroprotective steroid is administered before the thrombolytic agent is administered, such as up to one hour, up to two hours, up to two and a half hours, or up to three hours, or longer, before the thrombolytic agent is administered. In other embodiments, the thrombolytic agent is administered before the neuroprotective steroid is administered, such as up to one hour, up to two hours, or up to three hours, or longer, before the neuroprotective steroid is administered. In some embodiments, the neuroprotective steroid and thrombolytic agent are administered substantially simultaneously. In such embodiments, the neuroprotective steroid and thrombolytic agent may be administered in separate compositions by the same or different routes of administration or in a single composition comprising both the neuroprotective steroid and thrombolytic agent. In accordance with any of these embodiments, the neuroprotective steroid and/or thrombolytic agent may be provided in a composition that additionally comprises a pharmaceutically acceptable carrier or diluent, as discussed in more detail above.

In accordance with some embodiments, the methods may comprise administering more than one neuroprotective steroid. In such embodiments, the neuroprotective steroids may be administered in separate compositions by the same or different routes of administration (sequentially or substantially simultaneously) or in a single composition comprising two or more neuroprotective steroids. In accordance with some embodiments, the methods may further comprise one or more further administrations of the same or different neuroprotective steroid(s).

In accordance with some embodiments, the methods may comprise administering more than one thrombolytic agent. In such embodiments, the thrombolytic agents may be administered in separate compositions by the same or different routes of administration (sequentially or substantially simultaneously) or in a single composition comprising two or more thrombolytic agents. In accordance with some embodiments, the methods may further comprise one or more further administrations of the same or different thrombolytic agent(s).

In some embodiments, the thrombolytic agent is tPA and is administered at a dose of from 0.1 to 5 mg/kg body weight of the patient, including a dose of 0.9 mg or 1.1 mg/kg body weight. In some embodiments, the tPA is administered to achieve a peak plasma concentration of tPA of from about 0.2 to 70 μM, including from about 1.0 to 10 μM. For example, the tPA may be administered in a first priming dose of about 440 IU/kg body weight, followed by a continuous administration of about 440 IU/kg/hr for 12 hours. In other embodiments, the tPA is administered in accordance with protocols approved by the U.S. Food and Drug Administration for the treatment of acute ischemic stroke. For example, the tPA maybe provided in a composition formulated for intravenous administration to provide a dose of 0.9 mg/kg body weight of the patient, such as up to a total dose of 90 mg. The tPA may be administered over the course of one hour, with about 10% of the total dose administered as a bolus administration over the first minute

In some embodiments, the thrombolytic agent is streptokinase and may be provided in a composition formulated for intravenous administration, such as to provide a dose of from 1,500,000 to 2,400,000 IU. The streptokinase may be administered continuously as an infusion over the course of one hour or from 24 to 72 hours, as needed, with about 10% of the total dose (for the 24 to 72 hour infusion) administered as a loading dose over the first 30 minutes.

In some embodiments, the thrombolytic agent is urokinase and may be provided in a composition formulated for intravenous administration, such as to provide a dose of from 50,000 IU to 750,000 IU, including 500,000 or 300,000 IU. The urokinase may be administered as an infusion over the course of 1-12 hours, with an optional initial loading dose administered for the first 10 minutes and then a continuous dose infusion for the remaining time.

In some embodiments, the thrombolytic agent is desmoteplase and may be provided in a composition formulated for intravenous administration, such as to provide 90 μg/kg, such as up to 9,000 μg (or 9 mg). The desmoteplase can be administered as a single bolus over 1 to 2 minutes.

In accordance with any of the embodiments discussed herein, the thrombolytic agent may be administered within the first 24 hours after the injury or onset of injury, including within the first 12 hours, or within the first 6 hours. In some embodiments, the thrombolytic agent is tPA and is administered within the first 5 hours, within the first 4.5 hours, within the first 4 hours, or within the first 3 hours after the injury or onset of injury. In some embodiments, the thrombolytic agent is streptokinase and is administered within the first 12 hours, 6 hours or 4 hours after the injury or onset of injury. In some embodiments, the thrombolytic agent is urokinase and is administered within the first 12 or 4 hours after the injury or onset of injury. In some embodiments, the thrombolytic agent is desmoteplase and is administered within the first 3 to 9 hours after the injury or onset of injury.

In accordance with any of the embodiments discussed herein, the administration of the neuroprotective steroid(s) can constitute part of a therapy that comprises a two-level dosing regimen of the neuroprotective steroid, such as described, for example in U.S. Pat. No. 7,473,687, the entire contents of which are incorporated herein by reference in its entirety. In specific examples of such embodiments, the two-level dosing regimen may comprise a first period of a first hourly dose, followed by a second period of a lower hourly dose. In specific embodiments, the first hourly dose may be in the range of about 0.1 mg/kg to about 10 mg/kg, and in particular about 0.1 to about 7.1 mg/kg, while the second hourly dose may be in the range of about 0.05 mg/kg to about 5 mg/kg. In specific embodiments, the neuroprotective steroid is present in the composition such that it can be administered intravenously at 12 mg/kg per day for 3-5 days.

In some embodiments, the neuroprotective steroid(s) is administered in a constant dosing regimen, in which a constant total hourly infusion dose is administered over the course of treatment. In specific embodiments, the constant dosing regimen is provided for about 12, 24, 36, 60, 72, 84, or 120 hours, or for about 1 to 24 hours, about 12 to 36 hours, about 24 to 48 hours, about 36 to 60 hours, about 48 to 72 hours, about 60 to 96 hours, about 72 to 108 hours, about 96 to 120 hours, or about 108 to 136 hours.

In accordance with any of the embodiments discussed herein, the administration of the neuroprotective steroid(s) can be followed by a tapered administration of the neuroprotective steroid(s), such as described, for example in U.S. Pat. No. 7,473,687, the entire contents of which are incorporated herein by reference in its entirety.

In accordance with any of the embodiments discussed herein, the neuroprotective steroid may be administered within the first 24 hours after the injury or onset of injury, such as within less than 1 hour, within 1 hour, within 2 hours, within 3 hours, or within 4, 5, 6, 7, 8, 9, 10, 11, 12, 13, 14, 15, 16, 17, 18, 19, 20, 21, 22, 23, or 24 hours after the injury or onset of injury.

In accordance with any of the embodiments discussed herein, both the thrombolytic agent and the neuroprotective steroid may be administered within the first 24 hours, 12 hours, 9 hours, 6 hours, 5 hours, 4 hours, 3 hours, or less, after the injury or onset of injury, such as after ischemic stroke.

In accordance with any of the embodiments discussed herein, the composition(s) may be administered once or several times a day, and the duration of the treatment may be for a period of about 1, 2, 3, 4, 5, 6, 7 days or more. In specific embodiments, a daily dose can be administered in a single administration or in multiple administrations.

As discussed above, the methods may comprise administering the composition(s) by any route of administration, including, but not limited to, systemic injection, orally, nasally, parenterally, intravenously, intraperitoneally, intramuscularly, transdermally, buccally, subcutaneously, topically.

In some embodiments, the ischemic injury is associated with neurodegenerative reaction to injury or disease, traumatic brain injury (TBI), ischemic CNS injury, spinal cord injury, ischemic stroke, or anterior optic nerve ischemic injury. Thus, the patient may be a subject suffering from or at risk of developing ischemic injury, including a subject suffering from or at risk of developing any of these or similar conditions.

In some embodiments, the patient is a subject at risk of ischemic injury, such as ischemic stroke, such as a subject suffering from atherosclerosis or with a family history of heart disease, or smoker, or over age 55. See, e.g., www.strokecenter.org/patients/about-stroke/stroke-statistics. In other embodiments, the patient is at risk of another ischemic injury, such as a subject whose work, status or lifestyle places him/her at risk for ischemic injury, such as CNS injury or TBI, including athletes and soldiers. In accordance with specific embodiments, a patient at risk of ischemic injury is considered to be a subject who will be administered a thrombolytic agent, and is administered a neuroprotective steroid in order to reduce the risks of treatment with a thrombolytic agent. In such embodiments, the neuroprotective steroid may be administered “prophylactically,” i.e., before the onset or occurrence of ischemic injury or stroke, such as being administered as part of an ongoing therapeutic regimen.

Other embodiments include methods of reducing the risks of treatment with a thrombolytic agent, comprising administering a neuroprotective steroid or a pharmaceutically acceptable salt, ester or prodrug thereof to a patient who has been or will be administered a thrombolytic agent.

In specific embodiments, the neuroprotective steroid prevents or reduces neurovascular complications caused by delayed thrombolytic agent treatment of ischemic injury. For example, the neuroprotective steroid may prevent or reduce confluent hemorrhagic infarction, cerebral hemorrhage and/or blood-brain barrier (BBB) disruption that may be induced by a thrombolytic agent.

For example, the mechanisms underlying tPA's neurovascular complications are not fully understood. It has been suggested that they are a result of BBB disruption. Hamann, et al, J. Cereb. Blood Flow Metab., 1996, 16(6): 1373-8. Matrix metallopeptidases (MMPs), a family of zinc-binding proteolytic enzymes, play an important role in modulating BBB disruption after cerebral ischemia by degrading the major components of basal laminar around the BBB microvasculature. Bryce and Rosenberg, J. Cereb. Blood Flow Metab., 1998, 18(11): 1163-72. Animal and human studies have provided strong evidence linking MMP-9 induction and tPA-induced hemorrhagic transformation in ischemic stroke. Lapchak et al, Stroke, 2000, 31(12): 3034-40; Wang et al, Nat Med, 2003, 9(10): 1313-7; Cheng et al., Nat Med, 2006, 12(11): 1278-85; Pfefferkorn and Rosenberg, Stroke, 2003, 34(8): 2025-30. Human studies have indicated that stroke patients with higher pretreatment plasma levels of MMP-9 are more likely to experience cerebral hemorrhagic complications after tPA. Castellanos et al, Stroke, 2003, 34(1): 40-46; Montaner et al, Circulation, 2003, 107(4): 598-603; Castellanos et al, Stroke, 2004, 35(7): 1671-76. Therefore inhibition of MMP-9 may represent an important strategy to increase the safety of tPA thrombolysis.

As noted above, in specific embodiments, the neuroprotective steroid is progesterone or allopregnanolone. While not wanting to be bound by any theory, progesterone or allopregnanolone may reduce neurovascular complications that may be caused by tPA by inhibiting MMP-9 expression, inhibiting MMP-2 expression and/or attenuating blood-brain barrier disruption. Additionally or alternatively, progesterone may reduce the neurovascular complications that may be caused by thrombolytic agent treatment, such as tPA treatment, by increasing the expression of neuroserpin, a serine protease inhibitor (serpin) that is released from neurons and inhibits tPA.

In some embodiments, the neuroprotective steroid may extend the therapeutic window of thrombolytic treatment following ischemic injury. In specific embodiments, the neuroprotective steroid extends the reperfusion window of the thrombolytic agent following TBI or ischemic stroke. For example, in specific embodiments, the neuroprotective steroid may prevent or reduce cerebral hemorrhage induced by tPA when the tPA is administered to an ischemic stroke patient more than 3 hours after the stroke occurred, and may extend the therapeutic window to greater than 3 hours, such as to 4, 4.5, 5, 6, 7, 8 or 9 hours, or longer, after stroke. As another example, the neuroprotective steroid may prevent or reduce cerebral hemorrhage induced by streptokinase when the streptokinase is administered to an ischemic stroke patient more than 4 hours after the stroke occurred, and may extend the therapeutic window to greater than 4 hours, such as 5, 6, 7, 8 or 9 hours, or longer, after stroke. As another example, the neuroprotective steroid may prevent or reduce cerebral hemorrhage induced by desmoteplase when the desmoteplase is administered to an ischemic stroke patient more than 3 hours after the stroke occurred, and may extend the therapeutic window to greater than 3 hours, such as 4, 5, 6, 7, 8 or 9 hours, or longer, after stroke. In general, the neuroprotective steroid may extend the therapeutic window of thrombolytic treatment following ischemic injury by 50%, 100%, 150%, 200%, 250%, 300%, or longer.

In specific embodiments, the neuroprotective steroid, such as progesterone or allopregananolone, is administered within about 2 hours of ischemic stroke and the thrombolytic agent, such as tPA, is administered about 2.5 hours later, such as within about 4.5 hours of ischemic stroke, e.g., beyond its usual therapeutic window. In other embodiments the thrombolytic agent, such as tPA, is administered within about 3 hours of ischemic stroke, e.g., within its usual therapeutic window, and the neuroprotective steroid is administered simultaneously or sequentially.

In some embodiments, the methods and compositions described herein may prevent, reduce the risks of, or ameliorate one or more physiological events following neural injury, such as cerebral edema, cortical contusions, inflammatory response and excitotoxic response. In some embodiments, the methods and compositions described herein may reduce the duration of one or more such physiological events.

While not being bound by any theory, the methods and compositions described herein may exert any one or more of the following beneficial effects: (i) reduced neurodegeneration due to apoptosis; (ii) enhanced motor function, (iii) reduced loss of motor function, (iv) reduced or eliminated inflammation, (v) reduced loss of visual function, (vi) reduced damage from an inflammatory process, (vii) reduced or eliminated edema, (viii) reduced or eliminated ischemia, (ix) enhanced tissue viability, (x) enhanced neuronal proliferation, growth or differentiation, (xi) enhanced physical recovery, (xii) enhanced behavioral recovery and (xiii) reduced loss of general function. Again, while not being bound by any theory, the methods and compositions described herein, may, in specific embodiments, by reducing or eliminating the inflammatory response, substantially reduce brain swelling and/or reduce the amount of neurotoxic substances (e.g., free radicals and excitotoxins) that are released from the site of injury.

Further, while not being bound by any theory, the methods and compositions described herein may exert beneficial effects on any one or more of the following parameters associated with thrombolytic agent treatment, such as tPA treatment, following ischemic stroke: confluent hemorrhagic infarction, cerebral hemorrhage, blood-brain barrier disruption and tPA-matrix metalloproteinase interactions.

For example, in specific embodiments, the methods and compositions described herein may decrease edema in affected tissue, such as by at least about 15%, about 15% to 30%, about 30% to 45%, about 45% to 60%, about 60% to 80%, or about 80% to 95%, or greater. Additionally or alternatively, in specific embodiments, the methods and compositions described herein may reduce neuronal cell death, such as by increasing neuronal survival in treated patients by at least about 10%, about 10% to 20%, 20% to 30%, 30% to 40%, 40% to 60%, 60% to 80%, or greater, compared to a patient in a control group.

### VI. EXAMPLES

The following protocols are used in the examples:

Focal Embolic Model of Stroke:

A focal thromboembolic model of cerebral ischemia is induced in rats by middle cerebral artery occlusion (MCAO) via the fibrin-rich clots technique under isoflurane anesthesia. Surgical anesthesia is induced with isoflurane (5% induction, 2% maintenance, 700 mm N₂O, 300 mm O₂). Rats are cannulated with a femoral artery catheter for measurement of arterial blood gases, pH (Ciba-Corning 248 pH/blood gas analyzer), blood glucose, hematocrit and mean arterial blood pressure. The body temperature of the animals is maintained at 36.5±5° C. with a heating pad controlled by rectal thermistors. A closed cranial window is made in the right parietal skull (2 mm posterior, 6 mm lateral to bregma) for laser doppler probe placement. The rats are mounted on a modified stereotaxic frame equipped with a facemask. The right common carotid artery (CCA), the right internal carotid artery (ICA) and the right external carotid artery (ECA) are exposed by a ventral incision in throat. After surgical preparation of the right CCA and ligation of the pterygo-palatine artery, a PE10 catheter is inserted into the ECA. The tip of the catheter is placed close to the carotid bifurcation. To achieve thromboembolic occlusion of the right middle cerebral artery, 12 medium-sized fibrin rich clots suspended in phosphate with phosphate buffered saline (0.3 ml) are injected by a 1 ml syringe connected to the catheter.

Delayed tPA to Cause Hemorrhage:

To mimic delayed tPA thrombolysis in the clinical setting, a reproducible model of hemorrhagic transformation was developed. MCAO is induced by placing homologous blood clots into the MCA followed by delayed (e.g., at 4.5 hours post MCAO-induction) administration of recombinant tPA (10 mg/kg body weight) through the tail vein. This protocol results in significant hemispheric enlargement and hemorrhage.

Permanent Focal Ischemia by the Direct Ligation of Middle Cerebral Artery (MCA):

Permanent focal cerebral ischemia is induced in the rat by direct ligation of the MCA. Peri-operative procedures and physiological monitoring are done similarly to those for thrombotic stroke surgery. Briefly, under anesthesia, the left CCA is isolated through a ventral midline neck incision and ligated permanently with a 4-0 surgical silk ligature. The left temporoparietal region of the head is shaved and a 2-cm incision is made vertically between the orbit and the ear canal. The underlying temporalis muscle is excised, and under direct visualization, the right MCA is exposed through a 2-mm burr hole drilled 2 mm rostral to the fusion of the zygomatic arch with the squamosal bone. Drilling is performed under saline irrigation to the area throughout the procedure to prevent heat injury. The MCA is visualized where it crosses the inferior cerebral vein, which lies within the rhinal fissure. The site of the occlusion is proximal to the MCA bifurcation, but distal to the origin of the lenticulostriate arteries. Using fine forceps, the dura and arachnoid overlying the MCA is opened and cut. The MCA is cauterized and cut permanently to ensure recanalization using a bipolar electrocauterizer without damaging the brain surface. The temporalis muscle and skin is closed in layers, and rats allowed to recover from anesthesia on the heating pad.

Lesion Volume Assessment:

Brains are removed, frozen, and sectioned coronally at a thickness of 20 μm from approximately 12 mm (frontal cortex with sensorimotor cortex) to 5.0 mm (middle of hippocampus) anterior to the interaural line of the brain. Infarct size is examined in tissue sections at 12 rostrocaudal levels with intervals of 500 μm, by staining with Nissl (0.5% CV). Briefly, tissue sections are immersed in 70% and 95% ethanol for 5 minutes each and in 100% ethanol for 15 minutes, then stained for 5 minutes in filtered CV solution, and then briefly rinsed in double-distilled water. The infarct region is defined as an area with reduced Nissl staining or containing dark pyknotic-necrotic cell bodies. The infarct area (cortical, subcortical and total) is traced by using a scanner and an image analysis system (ImagePro™ by Media Cybernetics). Infarct volume is calculated by multiplying each sequential infarct area by the distance between sections and presented as a percentage of the volume of the contralateral hemispheric structure (cortex, caudate-putamen and total). This method of calculation and presentation of infarct volume eliminates and corrects the contribution of post-ischemic edema to the volume of injury.

Progesterone and Allopregnanolone Preparation and Administration:

All experimental treatments by injection are made in stock solutions using HBC (2-hydroxypropyl-β-cyclodextrin, 25% w/v solution in H₂O) as the solvent. The HBC vehicle allows progesterone and allopregnanolone to be dissolved in a non-toxic aqueous solution. The initial dose of progesterone is given intraperitoneally for rapid absorption; all other injections are given subcutaneously. A simlar protocol may be followed for allopregnanolone.

Plasma and Serum Sampling:

Blood is sampled from each animal just prior to decapitation. At each time point, animals are anesthetized according to approved decapitation protocol. Through a cardiac puncture, 1.5 ml of blood is withdrawn into syringes containing 0.2 ml of disodium citrate (0.16 mol/L) to determine coagulation rates. Another 0.5 ml of blood is withdrawn for the separation of serum. The samples are stored at −80° C. until further assay.

Hemoglobin Assay:

2,3,5-tripheynltetrazolium (TTC)-stained brain sections are used for quantification of intracerebral hemorrhage with a spectrophotometric hemoglobin assay. Samples typically are collected 19.5 hours after injury. The brain tissue is homogenized in 2 ml 0.05 M tris buffered saline and tween (TBS-T) followed by a 30-minute centrifugation (12,000 rpm) and re-centrifugation for 15-minutes at 10,000 rpm. Then, 200 μl reagent (QuantiChrom™ Hemoglobin Assay Kit; BioAssay Systems) is added to a 50 μl supernatant. Optical density is measured at 405 nm with a spectrophotometer.

Measurement of Brain Edema/Swelling:

Brain edema/swelling are estimated by measuring the hemispheric areas of each 2-mm thick brain slices using Image J software (NIH, Bethesda, Mass., USA), typically 24 hours after MCAO. Edema formation is calculated as hemispheric enlargement and expressed as a percentage of the normal areas in the contralateral hemisphere.

Cell Culture:

An immortalized mouse brain endothelial cell line (bEND 3) is purchased from American Type Culture Collection (ATCC, VA, USA) and cultured in Dulbecco's modified Eagle Medium (DMEM, ATCC) supplemented with 10% fetal bovine serum (ATCC). bEND 3 cells are seeded onto the luminal side of a filter (0.4 μm pore size; Corning, Lowell, MCA) coated with fibronectin (5 μg/ml) in 12-well plates for a transendothelial electrical resistance (TEER) test; and in 100-mm dishes for Western blot studies. Cell cultures are incubated at 37° C. in a 5%/95% mixture of CO₂ and atmospheric air, and the medium is replaced every 3 days.

Cell and Tissue Processing for Western Blotting:

Peri-infarct cortical tissue is processed for protein analysis. Tissues are homogenized in T-per (Pierce, Rockford, Ill.) containing protease inhibitor cocktail (P8340, Sigma). Homogenates are centrifuged for 15 minutes at 13,000 g. 40 μg of total protein is separated on 8-12% gel and transferred onto polyvinylidene difluoride (Millipore, Mass., USA; PVDF) membranes at 300 mA for 2 hours. To measure the level of immunoglobulin, the membranes were incubated overnight at 4° C. with anti Rat IgG conjugated to biotin. bEND 3 cells are subjected to hypoxia/reperfusion for 9 hours and then lysed on ice in 100 μl of RIPA buffer. Cell lysated are boiled and then electrophoresed in 12% SDS-PAGE acrylamide gels, transferred onto PVDF membranes, and incubated for 1 hour in TBS-T (tris-buffered saline and 0.1% Tween 20) containing 5% bovine serum albumin (BSA). Membranes are then incubated overnight at 4° C. with primary antibodies against occludin (Invitrogen, Calif., USA; 1:1000), claudin-5 (Invitrogen; 1:1000), and ZO-1 (Invitrogen; 1:1000), washed in TBS-T, and incubated for 1 hour at room temperature with corresponding peroxidase-conjugated secondary antibodies (1:5000; KPL, Gaithersburg, Md.). All blots are stripped and re-incubated with β-actin antibodies (1:5000, Sigma) as a loading control. The peroxidase reaction is developed with an ECL-plus detection kit (Amersham BioScience, Piscataway, N.J.). Intensity of the bands is measured by an Image Gauge version. 4.0 program (FUJI Photo Film Co., Ltd).

Western Blotting of Occludin in H/R-Treated bEnd 3 Cells:

The bEnd 3 cells grown to confluence on coverslips are subjected to the indicated treatments. For Western blotting, cells are washed three times with PBS, fixed in 3% paraformaldehyde for 10 minutes, permeabilized with 02% Triton X-100 in PBS for 10 minutes and blocked with 10% BSA and 0.1% Triton X-100 in PBS for 1 hour. After washing with PBS, the monolyers are incubated with a primary antibody for occludin (Invitrogen, 1:200) and MMP-9 (Abeam, Mass., USA, 1:200) overnight at 4° C. The cell monolayers are then washed 3 times with PBS and incubated with Alexa Fluor 488 anti-rabbit and orange anti-mouse (Molecular Probes, Eugene, Oreg.) for 1 hour. After washing in PBS, the coverslips are mounted on glass slides with Vectasheild with dapi (Vecta Laboratories).

Active Matrix Metalloproteinase-9 Assay:

A Fluorimetric SensoLyte™ 490 kit (AnaSpec Corp., San Jose, Calif. USA) is used to quantify the specific enzymetic activity of active MMP-9 by using a fluorescence resonance energy transfer peptide containing a fluorescent donor and quenching acceptor (Lin, Hou et al. 2008). The samples (100 μg) are placed in a 96-well plate containing 50 μl of assay buffer. The proteolytic resonance energy transfer peptide is expressed as a fold change in fluorescence intensity at excitation of 355 nm/emission of 460 nm.

The Measurement of TEER:

The electrical resistance across the membrane are measured using an Millicell ERS Voltohmmeter (Millipore, Billerica, Mass.). The extracellular matrix-treated Transwell inserts are placed in 12-well plates containing culture medium and then used to measure the background resistance. The resistance measurements of these blank filters are then subtracted from those of filters with cells. Values are shown as Q. based on culture inserts.

Behavioral Assays:

Motor coordination—accelerating rotarod: Motor impairment is assessed with the accelerating rotarod. Rats are given 3 training sessions 5 minutes apart before surgery. The animals are first habituated to the stationary rod, and then exposed to the rotating rod. The rod is started at 2 rpm and accelerated linearly to 20 rpm within 300 seconds. Latency to fall off the rotarod is determined before ischemia (presurgery) and postsurgery. Animals are required to stay on the accelerating rod for a minimum of 30 seconds. If they are unable to reach this criterion, the trial is repeated for a maximum of 3 times. The two best (largest) fall latency values a rat achieves are averaged and used for data analysis. Rats not falling off within 5 minutes are given a maximum score of 300 seconds. A sham-operated group is also included.

Gait analysis: On days 3, 7, 14, and 21 post-injury, animals are tested on the gait scan (Noldus CatWalk-automated gait analysis) apparatus. This consists of an enclosed walkway on a glass plate that is traversed by a rat from one side of the walkway to the other. Paw prints are captured by a high-speed video camera positioned under the walkway. Data generated include (1) spatial parameters related to individual paws (intensity, maximum area, print area, box width, box length, and paw angle); (2) relative spatial relationship between different paws (base of support, relative paw placement, and stride length); (3) interlimb coordination (step pattern, regularity index, and phase lag); and (4) the temporal parameters of gait (swing, stance, cadence, and walk speed).

Spontaneous locomotor activity: Digiscan™ activity-monitoring boxes are to assay spontaneous motor activity of the rats on pre-surgery day 5, and post-surgery times. Each session lasts 5 minutes and is conducted under red-light conditions. The following parameters are analyzed for each activity session for each rat: number of vertical movements, time spent in the center of box, time spent in the corners, and total horizontal distance transverse.

Grip strength: Animals are evaluated for the degree of force necessary to make the animal release a pull grid assembly by the forepaws using a grip strength meter.

Spatial navigation (MWM): The MWM test is reliably sensitive to unilateral lesions that tend to be much smaller than those produced by MCAO injury. The apparatus consists of a 133 cm-diameter circular tank filled with opaque water (20±1° C.) to a depth of 64 cm (23 cm from top of tank). A platform (11 cm×11 cm) is submerged to a depth of 2 cm and placed approximately 28 cm from the wall of the pool in the center of the northeast quadrant. Each trial is videotaped by a ceiling-mounted video camera and the animals' movement tracked using a computer-assisted tracking system. Two types of tests are performed: (1) acquisition of spatial memory and (2) spatial probe trial performed after the acquisition phase. Testing begins 10 days post-injury, and rats are examined for 10 days with 4 trials each session. For the spatial memory acquisition test, the performance of each rat is measured in terms of latency to platform, length of path to platform, and swim strategy, i.e., percent of total time spent in the outer versus inner annulus. On the eleventh session, rats undergo a “probe trial” in which the platform is removed and the rats are placed into the core of the pool and allowed to swim freely for 90 seconds. This task allows measurement of swim strategies as well as working (short-term, trial-to-trial), and reference (longer-term, day-to-day) memory. Time spent in the quadrant that previously contained the platform is recorded and calculated as percentage of total time spent in pool.

### Example 1

Progesterone and Allopregnanolone Attenuate MMP Induction in Ischemic Brain

Matrix metallopeptidases (MMPs), a family of zinc-binding proteolytic enzymes, play an important role in mediating BBB disruption after cerebral ischemia by degrading the major components of basal lamina surrounding the BBB microvasculature. Progesterone or allopregnanolone treatment following permanent ischemic stroke in rats, beginning approximately 1 hour post-occlusion, attenuated ischemia-induced MMP-9 and MMP-2 up-regulation.

Rats were randomly assigned to receive progesterone (n=5), allopregnanolone (n=5), or vehicle (n=5). Progesterone and allopregnanolone (8 mg/kg) were dissolved in 22.5% 2-hydroxypropyl-β-cyclodextrin (25% w/v solution in water) (HBC) and given by intraperitoneal injection 1 hour post-occlusion. An additional 8 mg/kg subcutaneous injection was given 6 hours after middle cerebral artery occlusion (MCAO).

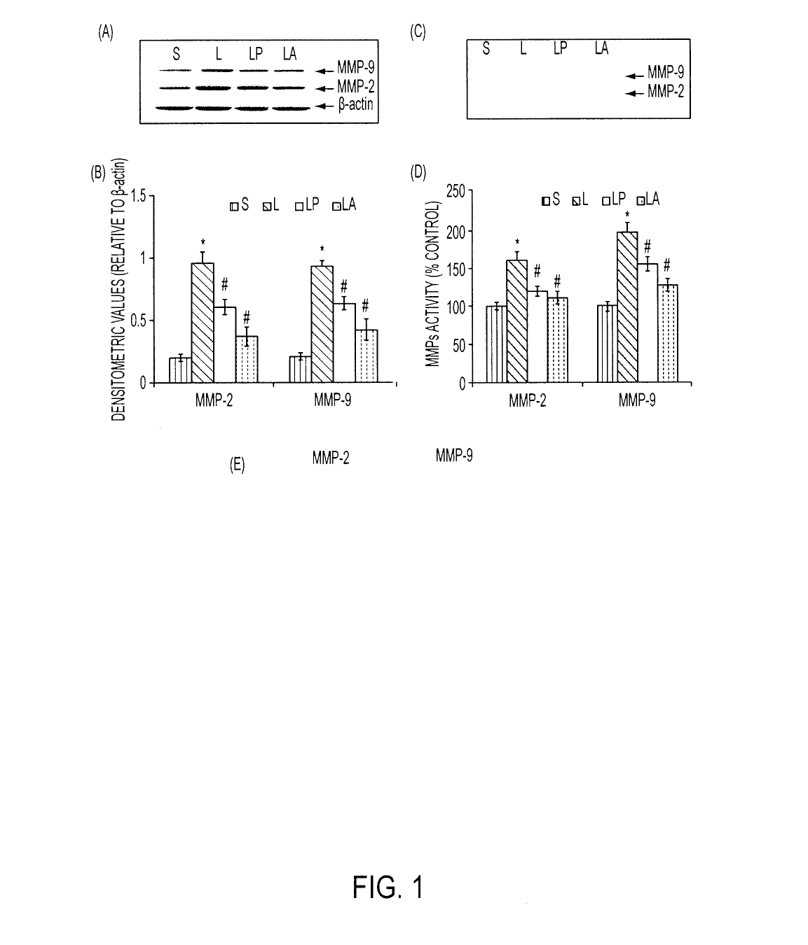

The rats were euthanized at 72 hours post-MCAO. The peri-infarct region was sampled using a 4-mm tissue punch. The results showed increased levels of MMP-9 and MMP-2 after permanent focal ischemia, indicating that progesterone and allopregnanolone attenuated MMP-9 and MMP-2 induction. Results are shown in FIG. 1.

FIG. 1A shows representative Western blots of MMP-2 and MMP-9 demonstrating regulation of their expression in peri-infarct cortex at 72 hours post-MCAO. β-actin is shown as a loading control. Expression of MMP-2 and MMP-9 was up-regulated after MCAO, and decreased by progesterone and allopregnanolone treatment.

FIG. 1B shows densitometric analysis of MMP-2 and MMP-9 (n=5) protein level in ischemic cortex after MCAO.

FIGS. 1C and 1D are gelatin zymography images showing the effects of progesterone and allopregnanolone on MMP-9 and MMP-2 activity in peri-infarct cortex. Representative gelatin zymogram and densitometric analysis indicates that MMP-9 and MMP2-levels were strongly enhanced at 72 hours after permanent MCAO, but were decreased by progesterone and allopregnanolone treatment.

FIG. 1E shows immunohistochemical examples of MMP-9 and MMP-2 expression in brain section. Expression of MMP-2 and MMP-9 immuno-positive signals were predominantly increased in the peri-infarct cortex at 72 hours after MCAO compared with sham-operated controls. Treatments with progesterone or allopregnanolone following pMCAO reduced the expression of MMP-9 and MMP-2.

### Example 2

Progesterone Prevents Cerebral Hemorrhage Induced by Tissue Plasminogen Activator Treatment Following Stroke

This example illustrates that progesterone can prevent delayed tPA treatment-induced cerebral hemorrhage and extend the reperfusion window in an experimental stroke model in rats.

Male Sprague Dawley (SD) rats were subjected to focal ischemia, reperfused at 4.5 hours and then treated with tPA (5 mg/kg body weight; via femoral vein) at 4.5 hours or by combined progesterone (given by intraperitoneal injection at 2 hours) plus tPA (given at 4.5 hours). Rats were killed at 24 hours after ischemia, brains were perfused with saline, and coronal sections (2 mm thick) were stained with 2,3,5-triphenyltetrazolium chloride to quantify infarct volumes. Cerebral hemorrhage was measured using a spectrophotometric assay to quantify hemoglobin in perfused brain.

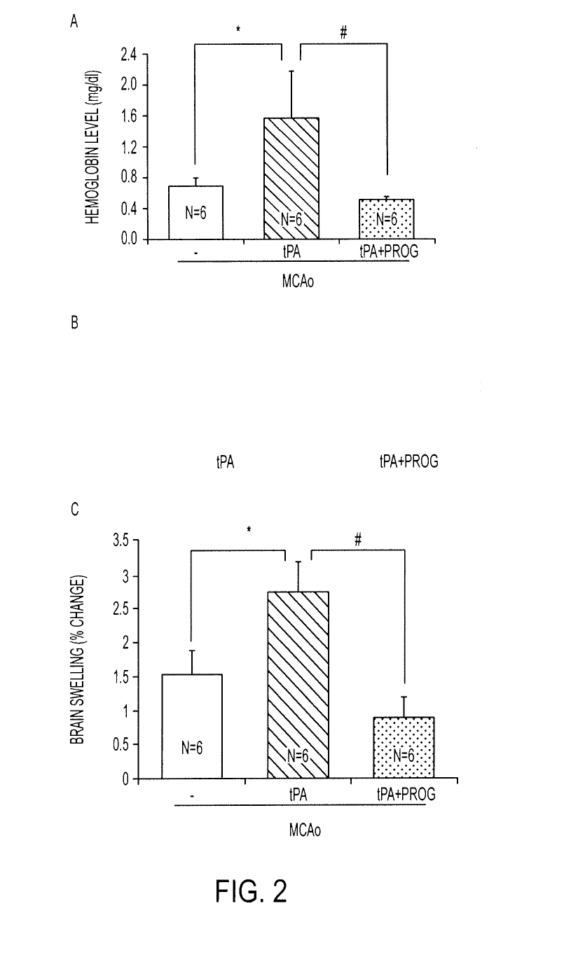

Results are shown in FIG. 2. FIG. 2A shows the effects of tPA and progesterone on hemorrhage volume at 24 hours post-stroke (mg/dl, expressed as mean±SE), and shows that delayed tPA (middle bar) induced cerebral hemorrhage, while combination therapy with delayed tPA plus progesterone (right bar) prevented hemorrhage (control bar on the left). FIG. 2B shows the effects of tPA and progesterone on infarct volume at 24 hours post-stroke, and shows that the combination therapy reduced infarct volume compared to tPA-only treatment. The reduction in hemorrhagic injury in combined progesterone and tPA-treated rats was 59.0% compared to rats treated with tPA but not progesterone. FIG. 2C shows the amount of cerebral edema (brain swelling, expressed as % change) caused by MCAO lesion, treatment with tPA (MCAO+tPA; middle bar) or combined treatment (PROG+tPA; right bar), indicating that the combined treatment results in a substantial reduction of tPA-induced cerebral edema (saline; left bar).

These data indicate that progesterone administration may extend the therapeutic time window for tPA administration in ischemic stroke, while at the same time reducing the risk of hemorrhagic conversion associated with tPA, and that the combined therapy will reduce the extent of brain injury following ischemic stroke.

### Example 3

Progesterone Prevents Blood-Brain Barrier (BBB) Disruption Induced by Treatment with tPA Following Stroke

This example shows that progesterone can prevent delayed tPA treatment-induced BBB disruption in both in vivo and in vitro models of BBB damage.

For in vivo evaluation, Male SD rats were subjected to MCAO, reperfused at 4.5 hours and then treated with tPA only (5 mg/kg body weight via femoral vein) at 4.5 hours post-stroke or with progesterone (intraperitoneally) at 2 hours post-stroke plus tPA at 4.5 hours post-stroke. Rats were killed at 24 hours post-ischemia and their brains perfused for evaluation of cerebral hemorrhage, brain swelling, BBB permeability, MMP-9 activity, and the alteration of tight junction proteins.

To mimic ischemia-like conditions in vitro, immortalized mouse brain endothelial (bEND3) cells were exposed to 6 hours of hypoxia and 3 hours of reoxygeneation with or without the administration of tPA at a dose of 20 μ/ml. Reoxygenation was initiated by adding glucose (4.5 g/L)-containing phenol red-free DMEM for 3 hours at 37° C. in 95% air and 5% CO₂. The cells were treated with various concentrations progesterone for 24 hours before hypoxia/reperfusion (H/R). BBB integrity was evaluated by transendothelial membrane electrical resistance (TEER) measurement, MMP-9 activity, and the alteration of tight junction proteins following in vitro H/R injury.

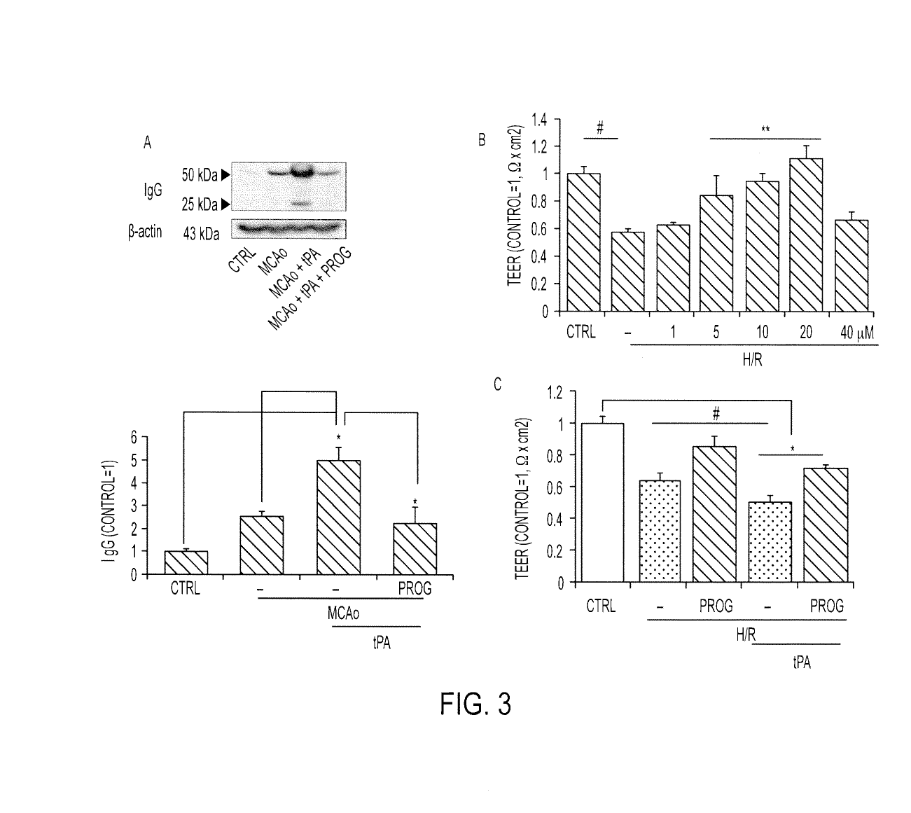

FIG. 3 depicts Western blot results for the in vitro study. To examine BBB disruption, the level of both the light (25 kDa) and heavy (50 kDa) chains of serum immunoglobulin G (IgG) were assessed at 24 hours after injury in the ischemic cortex. The results of Western blotting showed that the levels of IgG increased up to 2× at 24 hours after ischemic injury and tPA treatment (FIG. 3A). In contrast, administration of progesterone (8 mg/kg, s.c.) reduced the levels of serum IgG. Because BBB integrity is correlated with TEER, TEER measurements were performed after H/R. TEER values significantly decreased after H/R compared to values in normoxia (FIG. 3B). The administration of tPA significantly decreased TEER under H/R conditions (FIG. 3C). TEER values of tissue treated with progesterone (20 μM) were significantly higher compared to untreated tissue (FIG. 3C).

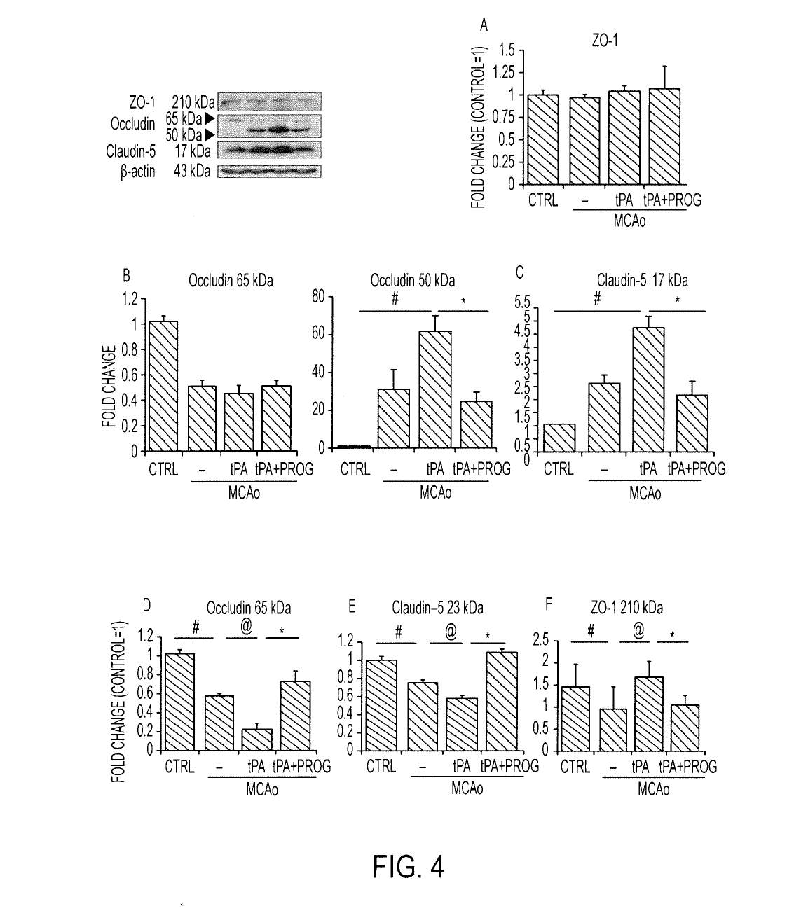

FIG. 4 depicts the effects of stroke with tPA and progesterone on three major tight junction (TJ) proteins (occludin, claduin-5, and ZO-1) involved in BBB permeability, using Western blotting for the in vivo (Figures A-C) and in vitro (Figures D-E) studies. For the in vivo study, the combination therapy with tPA plus progesterone significantly prevented the increase of 50-kDa occludin (FIG. 4B). Claudin-5 normally has a molecular weight of 22-kDa, but when it is degraded, lower molecular weight fragments are seen. The Western blot demonstrates an increase the lower molecular weight 17-kDa claudin-5 after stoke with tPA compared to stroke without tPA. In contrast, the administration of combined progesterone and tPA treatment significantly prevented the increase of lower molecular weight fragments in the ischemic cortices (FIG. 4C).

For the in vitro study, application of tPA with H/R markedly decreased the expression of occludin and claudin-5. The addition of progesterone significantly prevented the decrease of occludin (P=0.02) and 23-kDa claudin-5 (P=0.000) levels (FIGS. 4D-E). However, there were no significant differences among all groups for ZO-1 (FIG. 4F).

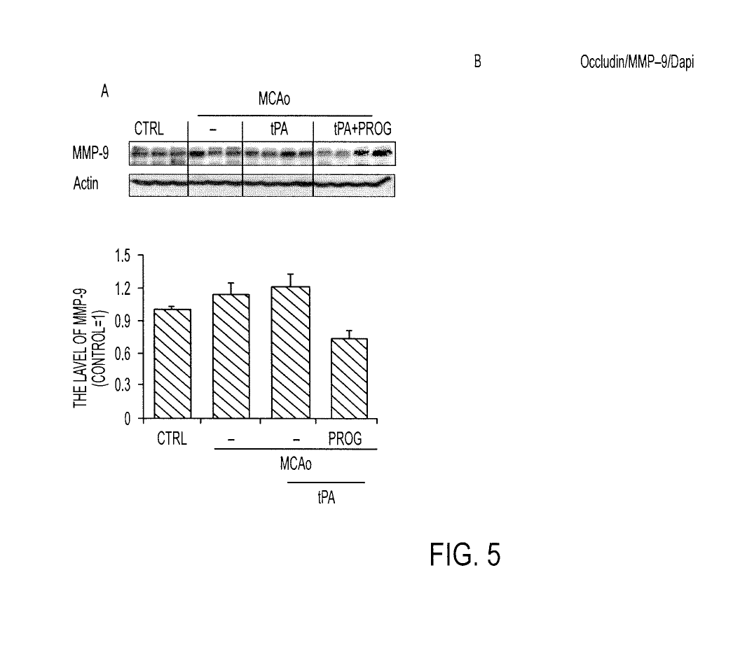

To directly determine MMP-9 activity after ischemia, proteolytic activity of MMP-9 was measured using a fluorescence resonance energy transfer peptide. MMP-9 activity increased at 24 hours after ischemia, especially in the group treated with tPA at 4.5 hours, which exhibited MMP-9 activity significantly higher than those in the control and transient middle cerebral artery occlusion (tMCAO) groups (FIG. 5A). To assess the possibility that MMP-9 is involved in H/R-induced disturbance in occludin expression, immunofluorescence staining was done to visualize the changes of occludin and MMP-9 in H/R with and without tPA-treated cells and progesterone. Consistent with the Western blot results, exposure of cells to H/R and tPA for 9 hours reduced the detected levels of occludin, as compared to control bEND 3 cells, while MMP-9 levels were increased in cells exposed to H/R and tPA for 9 hours. These effects were completely inhibited when cells were pretreated with progesterone (FIG. 5B).

These findings indicate that combination treatment with the anti-thrombotic agent tPA and progesterone is useful for decreasing the risk and severity of thrombolytic-associated BBB dysfunction leading to hemorrhage.

### Example 4

Effects of Progesterone and Allopregnanolone on the Expression of Coagulation Factors that can Enhance or Impair Recovery from Ischemic Stroke

This study will examine systemic and brain coagulation following ischemic stroke by focusing on clotting time and coagulation cascade proteins measured in the penumbral area of injury following progesterone and allopregnanolone treatment, including the following: CF-II (prothrombin), coagulation factor XIIIa, fibrin, neuroserpin and endogenous tPA.

Rats will be assigned to one of five survival time points and to one of three treatment groups: vehicle, progesterone or its metabolite allopregnanolone. The rats will undergo embolic MCAO (as described above) followed by either progesterone (8 mg/kg body weight), allopregnanolone (8 mg/kg body weight), or vehicle (HBC) injections at 2, 6, 24, 48, 72, 96 and 120 hours post-occlusion, with the number of injections determined by the scheduled survival time: 6, 24, 48, 72, or 120 hours post-occlusion. At 2 hours post-MCAO, physiological saline (2.5 mL/kg body weight) with or without recombinant tPA (10 mg/kg body weight) will be administered (10% bolus, 90% continuous infusion) to rats through the tail vein. Physiological monitoring will be performed as described above. Rats will be sampled for rtPCR and Western blot analysis of specific hemostatic protein levels and coagulation assays.

Blood will be sampled from each animal just prior to decapitation. The rats will be fatally anesthetized, decapitated, and their brains removed and sectioned for rtPCR analysis and Western assays. Total RNA will be extracted from fresh tissue using brain punches for all experimental groups, using the TRIzol reagent protocol (Life Technologies). Real-time PCR will be performed using primers designed by Primer Express software (Applied Biosystems) for the proteins of interest (tPA, neuroserpin, thrombin, and cFXIII). Antibodies against thrombin, CF XIII, fibrin, tPA, and neuroserpin will be used for Western blot analyses of brain tissue for all treatment groups and time points.

The assay for prothrombin (PT) and activated partial thromboplastin (APTT) will be performed on an automated fibrometer. PT will be determined by mixing plasma with a thromboplastin reagent and timing the formation of the initial clot. APTT will be determined by mixing sample plasma with 25 mmol calcium chloride/L and a partial thromboplastin reagent (Sigma) and timing initial clot formation.

This study is expected to show that progesterone increases the expression of coagulation factors in the ischemic brain, while allopregnanolone does not. In particular, this study is expected to show that progesterone reduces the expression of tPA by increasing the expression of neuroserpin. Further, this study is expected to show that the presence of fibrin is correlated with the expression of procoagulant factors, and negatively correlated with tPA.

### Example 5

Effects of Progesterone and Allopregnanolone on Hemorrhagic Transformation after tPA Therapy in a Rat Thromboembolic Model of Stroke

The effects of progesterone and allopregnanolone on infarction, cerebral hemorrhage, and blood-brain barrier (BBB) integrity following tPA administration will be determined, and the underlying mechanisms of the effects of progesterone and allopregnanolone on attenuating neurovascular complications associated with tPA will be examined by looking at tPA-MMP interactions after embolic ischemic stroke with and without tPA treatment. Delayed tPA administration (beyond the established 3-3.5 hour window) reproducibly causes hemorrhagic transformation, allowing study of the effects and mechanisms of progesterone and allopregnanolone in attenuating hemorrhagic damage.

Five groups of animals (n=26 in each group) will be compared: (1) sham controls administered vehicle (HBC); (2) ischemic controls administered vehicle (HBC); (3) rats administered tPA at 6 hours post-surgery (10 mg/kg; 1 mg/1 ml in saline); (4) rats administered progesterone (8 mg/kg body weight) and tPA at 6 hours post-surgery; and (5) rats administered allopregnanolone (8 mg/kg body weight) and tPA at 6 hours post-surgery. For the sham and non-tPA treatment groups, saline (10 ml/kg, intravenous) will also be administered 6 hours post-surgery. Rats will undergo embolic stroke (MCAO) followed by treatment according to their group.

This study is expected to show that progesterone or allopregnanolone treatment, by inhibiting MMP-9 and MMP-2 expression and attenuating BBB disruption, will reduce the neurovascular complications associated with delayed tPA thrombolysis after thrombotic stroke.

### Example 6

Behavioral Recovery after Post-Injury Treatment with tPA Combined with Progesterone or Allopregnanolone in a Thrombotic Model of Stroke in Rats

This study will assess the effects of a combination neuroprotective steroid and tPA treatment on cognitive/spatial performance in the Morris Water Maze (MWM) task, motor performance on the rotorod and foot-fault tasks, and metabolic homeostasis.

Rats will be divided into treatment groups corresponding to tPA alone or with progesterone or allopregnanolone and/or vehicle (control). Two hours post-injury (MCAO), rats will receive 8 mg/kg intraperitoneal injections of progesterone, allopregnanolone or vehicle. Subcutaneous injections will then be given at 6, 24, 48, 72, 96, 120, 144 (tapered dose) and 168 hours (tapered dose) post-injury. Tapering will be induced as halved dosages over the last two days of treatment. tPA (10 mg/kg; 1 mg/1 ml in saline) will be administered 2 hours post-occlusion by injection pump over a period of 30 minutes. For the non-tPA-treatment groups, saline (10 ml/kg) will be administered 2 hours after MCAO.

Animals will be tested pre-injury to establish baseline performance on spontaneous motor behavior (a measure of habituation/hyperactivity), grip strength, and the rotarod, and then retested at specific times post-injury. The rats will also be tested for foot fault and cognitive and spatial navigational performance on the MWM starting on day 10 post-injury.

The study is expected to show that the administration of progesterone or alllopregnanolone in combination with tPA after ischemic stroke leads to better and sustained behavioral recovery on a battery of measures sensitive to stroke-induced deficits.

The foregoing detailed description is intended to illustrate the various embodiments for methods described herein. As such, this detailed description is not meant to be limiting of the scope or application of the embodiments listed herein. It will be understood by persons skilled in the art that numerous modifications, substitutions, changes, or replacements with equivalents may be made to the particulars of the disclosure without altering the scope of the embodiments, and that such equivalents are to be included herein.

What is claimed is:

1. A method of treating a patient suffering from or at the risk of ischemic injury, comprising administering to said patient: (i) a neuroprotective steroid or a pharmaceutically acceptable salt, ester or prodrug thereof, and (ii) a thrombolytic agent, wherein the steroid is a progestogen.

2. The method of claim 1, wherein said progestogen is selected from the group consisting of progesterone and allopregnanolone.

3. The method of claim 1, wherein said thrombolytic agent is selected from the group consisting of tPA, streptokinase, urokinase and desmoteplase.

4. The method of claim 1, wherein said steroid and thrombolytic agent are administered in separate compositions.

5. The method of claim 1, wherein said steroid and thrombolytic agent are administered in the same composition.

6. The method of claim 1, wherein said steroid and thrombolytic agent are administered simultaneously.

7. The method of claim 1, wherein said steroid and thrombolytic agent are administered sequentially.

8. The method of claim 7, wherein said steroid is administered before said thrombolytic agent is administered.

9. The method of claim 1, wherein the ischemic injury is associated with a condition selected from the group consisting of a neurodegenerative reaction to injury or disease, traumatic brain injury, ischemic CNS injury, spinal cord injury, ischemic stroke, and anterior optic nerve ischemic injury.

10. The method of claim 9, wherein the ischemic injury is associated with ischemic stroke.

11. A method of reducing the risks of treatment with a thrombolytic agent, comprising administering a neuroprotective steroid or a pharmaceutically acceptable salt, ester or prodrug thereof to a patient who has been or will be administered a thrombolytic agent, wherein the steroid is a progestogen.

12. A pharmaceutical composition comprising:
(a) a neuroprotective steroid or a pharmaceutically acceptable salt, ester or prodrug thereof, wherein the steroid is a progestogen; and
(b) a thrombolytic agent, optionally, in a pharmaceutically acceptable carrier.

13. The composition of claim 12, wherein said propestogen is selected from the group consisting of progesterone and allopregnanolone.

14. The composition of claim 12, wherein said thrombolytic agent is selected from the group consisting of tPA, streptokinase, urokinase and desmoteplase.

15. The composition of claim 12, formulated for intravenous administration.

16. A pharmaceutical composition comprising a neuroprotective steroid or a pharmaceutically acceptable salt, ester or prodrug thereof, optionally, in a pharmaceutically acceptable carrier, for use in reducing the risks of treatment with a thrombolytic agent, wherein the steroid is a progestogen.

17. The pharmaceutical composition of claim 16, wherein said progestogen is selected from the group consisting of progesterone and allopregnanolone
